(19) United States
(12) Patent Application Publication
(10) Pub. No.: US 2017/0325825 A1
(43) Pub. Date: Nov. 16, 2017

(54) Smart Tourniquet

(71) Applicant: Carebay Europe Ltd, Sliema (MT)

(72) Inventor: Farhad Bybordi, Boynton Beach, FL (US)

(73) Assignee: Carebay Europe Ltd, Sliema (MT)

(21) Appl. No.: 15/533,017

(22) PCT Filed: Oct. 23, 2015
(86) PCT No.: PCT/EP2015/074578

§ 371 Date: Jun. 3, 2017

(30) Foreign Application Priority Data
Dec. 4, 2014 (SE) 1451480-6

(51) Int. Cl. A61B 17/135; A61B 5/15; A61M 5/42; A61B 5/00; A61B 5/024; A61B 17/00

(52) U.S. Cl. CPC A61B 17/1355; A61B 5/15003; A61B 5/150076; A61B 5/150129; A61M 5/422; A61M 5/427; A61B 5/02438; A61B 5/489; A61B 5/150748; A61B 2017/00115; A61B 2017/00199; A61B 2017/00734; A61B 2017/00022

(57) ABSTRACT

A smart tourniquet includes a casing having a control unit; a contact area arranged to the casing, configured to contact a patient's skin, and connected to the control unit; a cuff arranged to the casing with an inflatable bladder; an adjustable strap arranged to the cuff for securing the cuff and casing to the patient; a pump contained in the casing, connected to the bladder, and controlled by the control unit; a thermoelectric module contained in the casing, controlled by the control unit, and connected to the contact area; and at least one sensor contained in the casing for detecting blood pulse and controlled by the control unit. The control unit is configured to inflate and deflate the bladder in response to blood pulse for changing a pressure around an arm or leg and to heat the contact area for vasodilating a vein under the patient's skin for visual detection.

### FIELD OF THE INVENTION

The present invention relates to a smart tourniquet arrangement which facilitates venipuncture operations. In particular, the invention relates to a portable device comprising features for locating veins, for reducing pain and for alleviating bleeding.

### BACKGROUND OF THE INVENTION

Every year the number of venipuncture operations increase thanks to scientific advances and improved methods and equipment for blood sample analysis. Many routine investigations performed by physicians require blood samples. Typically, when blood is drawn for lab analysis, a caregiver/physician needs to locate a suitable vein. Normally, the caregiver/physician fastens an uncomfortable rubber tourniquet on a patients arm in order to obstruct venous blood flow such that the veins expand and show more clearly under the skin of the patient. Often the caregiver/physician must carefully locate a suitable raised vein followed by cleaning of the insertion site. The procedure is the same for infusions for the purpose of drug administration or any type of fluid IV therapy.

Upon completion of blood collection or infusion, proper practice calls for application of pressure on the needle insertion site by the patient or by the caregiver/physician for a period of time, and if the patient has a blood disorder disease, like Haemophilia, then the application of pressure is combined with cooling patches and soothing pressure for an extended period of time in order to prevent further bleeding.

Sometimes during this procedure, a suitable vein proves difficult to locate and it is often difficult to reliably insert a needle because the walls of the patient's vein have degenerated at the site of venipuncture either from the effects of previous punctures, because the patient is elderly or obese, and/or because the patient's vein is prone to roll from side to side. Thus, non-reliable and repeated venipuncture causes discomfort and accumulative injury to the patient and may result in poor collection of blood and/or drug administration.

Devices for stimulating veins in order to improve the process of venipuncture are known in the prior art.

U.S. Pat. No. 4,834,802 discloses a disposable heat-generating tourniquet for stabilizing and vasodilating a vein for venipuncture. The vein is stabilized by physically locking, or immobilizing it within a window of a tourniquet pad portion of the device. The simultaneous vasodilation is both by mechanical means and also by thermal means. The device causes the vein to pressurize and to swell.

U.S. Pat. No. 3,620,209 discloses a device which is held against the skin, nominally by straps, and which vibrates the skin about the injection area. The vibration stimulates the pain center of the skin so that the pain of injections perceived by the patient may be minimized.

The present invention seeks to provide an improved smart tourniquet arrangement that helps caregivers/physicians and/or patients to locate veins or arteries and which provides various advantages over currently known devices.

### SUMMARY OF THE INVENTION

According to a first aspect of the present invention, there is provided a smart tourniquet arrangement for use in venipuncture of intravenous insertions comprising: a casing having a control unit; a contact area arranged to the casing, the contact area being constrained to contact a patient's skin and connected to the control unit. The arrangement further comprises a cuff arranged to the casing and having an inflatable bladder; an adjustable strap arranged to the cuff for securing the cuff and the casing to the patient; a pump contained within the casing, said pump being connected to the inflatable bladder and being controlled by the control unit; a thermoelectric module contained within the casing, said thermoelectric module being controlled by the control unit and being connected to the contact area; sensors contained within the casing for detecting blood pulse, the sensor being controlled by the control unit. The smart tourniquet arrangement of the invention comprises the control unit configured to inflate and deflate the inflatable bladder on response to blood pulse for increasing or decreasing an amount of squeeze around an arm or around a leg and, to heat the contact area for vasodilatating the veins, causing the veins to be exposed and surfaced on to the patient's skin for visual detection.

The smart tourniquet arrangement has a variety of uses combined in one refined system which allows the patient and/or caregiver/physician to feel more confident in the task of venipuncture operations. The smart tourniquet arrangement is applied comfortably, easily, and quickly to the patients arm or to some other suitable site. Inflation and deflation of the arrangement is useful for applications involving the elderly and children since the pressure of a tourniquet (typically a simple uncomfortable rubber strap) can cause some pain to patients belonging to those groups. The arrangement uses a reliable comfortable method to tighten the tourniquet around the arm or around the leg in order to create the necessary pressure to raise the veins to become more pronounced in order to provide a better visual indication of a suitable vein for needle insertion.

According to another aspect of the present invention, the control unit is also configured to cool the contact area causing a thermal analgesic effect which reduces pain. The cooling effect also constricts the veins whereby bleeding may be reduced.

According to yet another aspect of the present invention, the contact area is located either on an outer surface of the casing or on the adjustable strap.

According to yet a further aspect of the present invention, the arrangement further comprises a vein locator assist system which is controlled by the control unit and comprises a portion which protrudes from the casing. The arrangement is a combination of several individual systems which are combined into one easy-to-use system. Thus, this vein locator assist system assists a caregiver/physician by providing a quick approach to finding any usable vein/artery which may normally not be detected via tactile or visual inspection. This is particularly beneficial for dark-skinned and/or obese patient, for whom standard techniques for vein/artery location are more difficult.

The tourniquet is made out of a soft washable fabric which is flexible and stretchable, allowing the caregiver/physician to relocate the tourniquet in any direction after it has been placed around arm or around a leg.

According to another aspect of the present invention, the casing comprises a receiving element for receiving a pad which can be externally heated or cooled. The pad may be a hydrogel pad. The arrangement provides a pocket which will allow the caregiver/physician and/or the patient to position the heated pouch/pad prior to any needle insertion in order to cause the area of needle insertion of the skin to warm up. This action causes the veins to swell and to fill with more blood, and consequently allows the veins to be raised and surfaced. It also provides the patient with the possibility to replace the heating pad with a cooling pad/pouch after the injection or infusion, since cooling and soothing of the area of needle insertion causes the vein to constrict and reduces further bleeding. This may be very beneficial for patients having blood disorder diseases such as Haemophilia. The user may also use an external thermal source instead of the thermoelectric element in case of discomfort.

According to yet another aspect of the present invention, a display and/or visual indicators and/or audio indicators and/or switch buttons is/are arranged on the casing and connected to the control unit. This provides a feedback to the caregiver/physician and/or the patient about the pressure of the inflated tourniquet arrangement and also allows the user to control the pressure and the thermoelectric module. It may be possible to increase or to decrease the pressure, as well as to switch from heating to cooling, or to switch off the thermoelectric module by manually operating the switch buttons. The caregiver/physician and/or patient can easily adjust the pressure on his/her arm as desired which may be especially beneficial to patients with fragile skin type and to young children.

According to a further aspect of the present invention, the arrangement further comprises a communication module configured to transmit and/or receive information and/or signals directly and/or indirectly from the control unit to a smart device. The arrangement allows the caregiver/physician and/or the patient to utilize a WI-FI and/or Bluetooth and/or a mobile communication network capability to connect with any other smart device. This is very useful especially if the patient is ambidextrous or has an amputated arm, therefore a caregiver/physician can trigger and control the different functionalities of the arrangement remotely.

According to another aspect of the present invention, the arrangement further comprises a storage case for storing additional items which is advantageous if the patient utilizes the arrangement for infusion and helps him/her to carry important items such as a syringe, needles, pads, etc.

The present invention seeks to provide an improved tourniquet arrangement which is an essential and required element in order to properly and completely administer a good and clean blood sample or infusion therapy.

The present invention resolves many of the difficulties encountered by prior art systems by providing a solid improvement and idea to make the tourniquet arrangement much more efficient and comfortable than a simple rubber strap.

### BRIEF DESCRIPTION OF THE DRAWINGS

Embodiments of the present invention will now be described by way of example with reference to the accompanying drawings in which:

FIG. 1 shows a perspective view of a smart tourniquet arrangement for use in venipuncture of intravenous insertions according to one embodiment of the invention;

FIG. 2 shows a top view of the embodiment of FIG. 1;

FIG. 3 shows a bottom perspective view of the arrangement of the invention from below;

FIG. 4 shows a perspective view of the arrangement of the invention according to FIG. 1 from a different angle;

FIG. 5 shows an exploded view of the arrangement of the invention according to FIG. 1;

### DETAILED DESCRIPTION OF THE INVENTION

The smart tourniquet arrangement (1) according to the invention may be used on any part of the body (e.g. arm, wrist) when venipuncture is required. An aim of the invention is to localize the vein or artery and to facilitate the procedure of intravenous (IV) insertion. The tourniquet arrangement is divided into three main parts: a casing (2), a cuff (7) and an adjustable strap (8). The adjustable strap (8) may be manufactured of different materials (e.g. cloth or synthetic fabric) and with different strap mechanisms (e.g. Velcro, buckle).

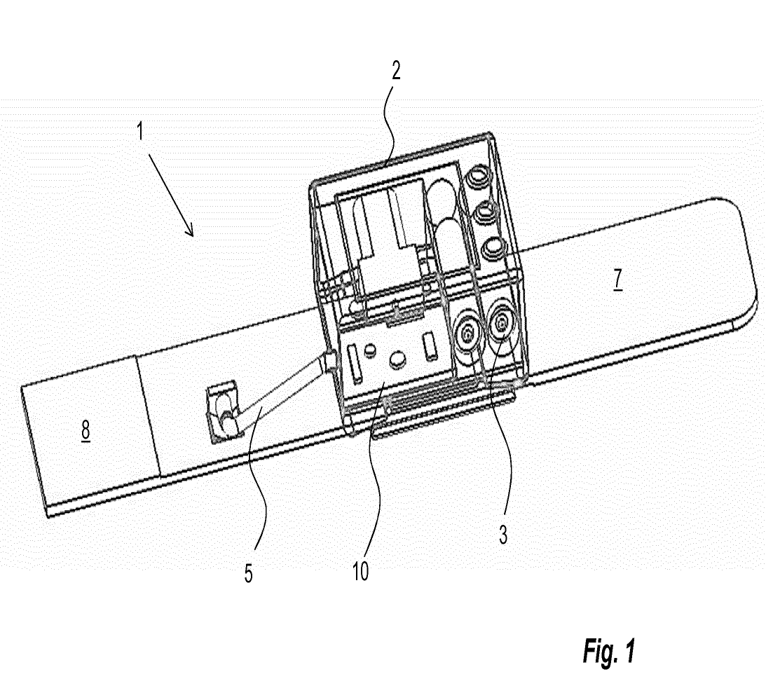

The casing (2) of the smart tourniquet arrangement has a control unit (10). The control unit (10) is equipped with a processor. The control unit (10) is defined as a component of a computer's central processing unit (CPU) that directs operations of the processor. It controls the computer's memory and arithmetic/logic unit and input and output devices and responds to a program's instructions (FIG. 1, 5).

There are different known techniques for measuring a patient's blood pressure. According to the invention, the cuff (7) is arranged to the casing (2) and the cuff (2) comprises an inflatable bladder (not shown). The cuff (7) is for example wrapped around the patient's upper arm and the inflatable bladder (15) is inflated to restrict blood flow through the patient's brachial artery. The cuff (7) of the invention may further comprise a manometer, a sleeve and tapered sections (not shown). Additionally, the adjustable strap (8) is arranged to the cuff for securing the cuff and the casing (2) to the patient. The tightening of the strap against the skin permits accurate measurements on the patient's body.

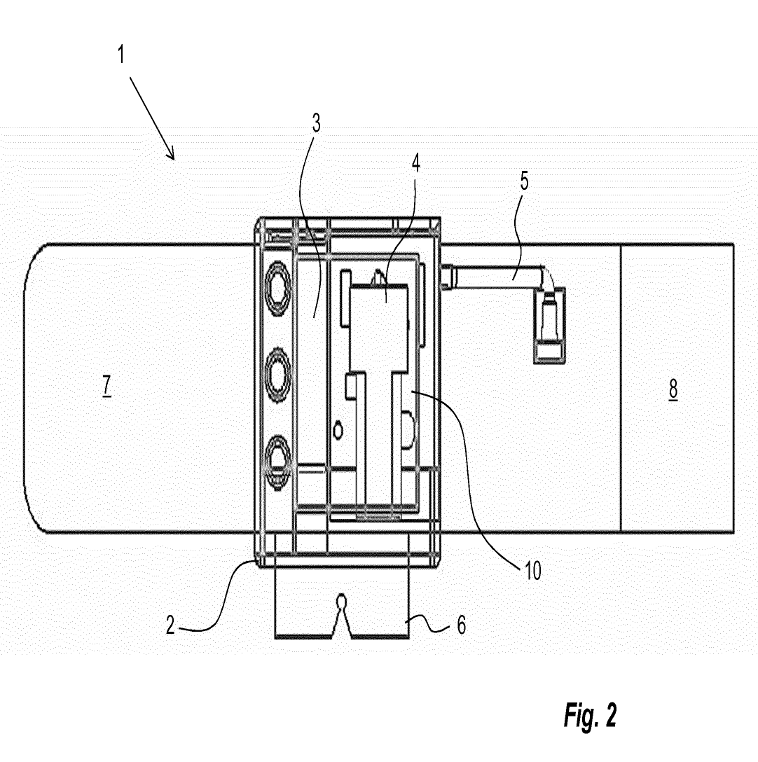

The arrangement of the invention further comprises a mini-pump (4) contained within the casing (2), said pump (4) is connected to the inflatable bladder (15) via a conduit element, such as a flexible tube (5) and the pump is controlled by the control unit (10). Hence, the control unit (10) is able to determine the amount of fluid (e.g. air) needed to inflate and deflate the inflatable bladder (15) in response to bladder pressure for increasing or decreasing the pressure on around the arm or around the leg preventing any harm to the patient's skin. At least one power supply (3), such as a battery, provides electric energy to the control unit (10) and to the mini-pump (4) (FIG. 2, 5). The arrangement is not limited to traditional batteries, but other energy sources can be used as well, e.g. solar cells.

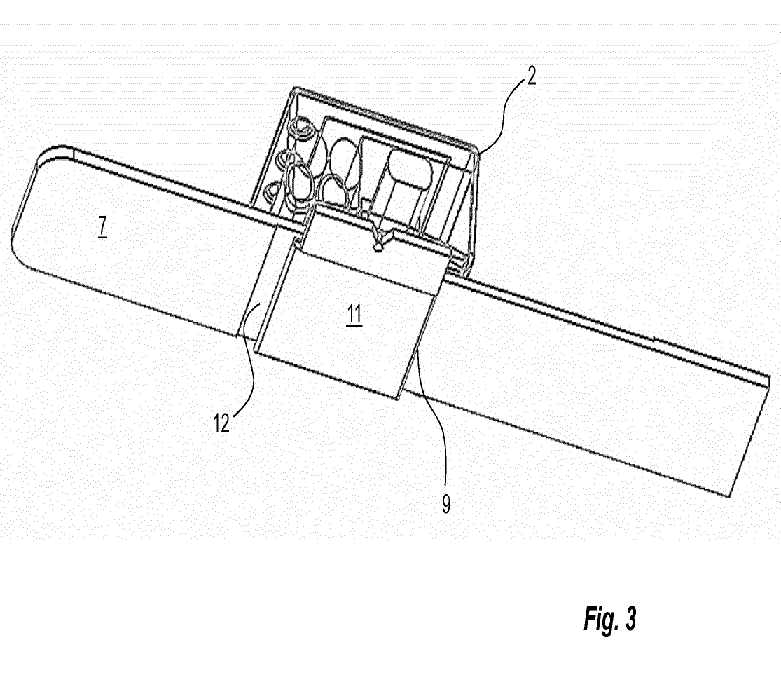

As illustrated in FIG. 3, the arrangement further comprises sensors (not shown) and a contact area (11) arranged to the casing. The contact area (11) is configured to contact the patient's skin and is connected to the control unit (10), which collects data from the sensors. The sensors may detect blood pulse and other relevant signals (e.g. HTD piezo electric). Thus, the sensors are controlled by the control unit (10).

A thermoelectric module (12) is contained within the casing (2) of the invention, the thermoelectric module (12) is controlled by the control unit (10) and connected to the contact area (11) in order to heat the contact area for vasodilatating the veins, causing the veins to swell and surface under the patient's skin for visual detection (FIG. 3, 5).

By applying a low voltage DC power to the thermoelectric module (12), heat will be transferred through the module from one side to the other. Therefore, one module face will be cooled while the opposite face is simultaneously heated. It is important to note that this phenomenon may be reversed. A change of the polarity (plus and minus) of the applied DC voltage will cause heat to be transferred in the opposite direction. Consequently, a thermoelectric module (12) may be used for both heating and cooling, thereby making it highly suitable for application requiring precise temperature control. A practical thermoelectric module (12) generally consists of two or more elements of n and p-type doped semiconductor materials that are connected electrically in series and thermally in parallel. These thermoelectric elements and their electrical interconnects are typically mounted between two ceramic substrates. The substrates hold the overall structure together mechanically, and electrically insulate the individual elements from one another and from external mounting surfaces. Most thermoelectric modules range in size from approximately 2.5-50 mm (0.1 to 2.0 inches) square and 2.5-5 mm (0.1 to 0.2 inches) in height. A variety of different shapes, substrate materials, metallization patterns and mounting options may be provided.

Both N-type and P-type as e.g. Bismuth Telluride thermoelectric materials are used in a thermoelectric cooler. This arrangement causes heat to move through the cooler in one direction only while the electrical current moves back and forth alternately between the top and bottom substrates through each N and P element. N-type material is doped so that it will have an excess of electrons and P-type material is doped so that it will have a deficiency of electrons. The extra electrons in the N material and the “holes” resulting from the deficiency of electrons in the P material are the carriers which transfer the heat energy through the thermoelectric material. Most thermoelectric cooling modules are fabricated with an equal number of N-type and P-type elements where one N and P element pair form a thermoelectric “couple.” For example, the module illustrated above has two pairs of N and P elements and is termed a “two-couple module”. Cooling capacity (heat actively pumped through the thermoelectric module) is proportional to the magnitude of the applied DC electric current and the thermal conditions on each side of the module. By varying the input current from zero to maximum, it is possible to regulate the heat flow and control the surface temperature.

The casing (2) further comprises a receiving element (9) for receiving a removable pad, e.g. a hydrogel pad The hydrogel pad may be removed from the receiving element for heating or cooling, i.e. by a microwave heater or a fridge, respectively (FIG. 3, 5). This can be used in case that the patient/user feels some discomfort using the thermoelectric module. The receiving element (9) may not be limited to receiving a pad.

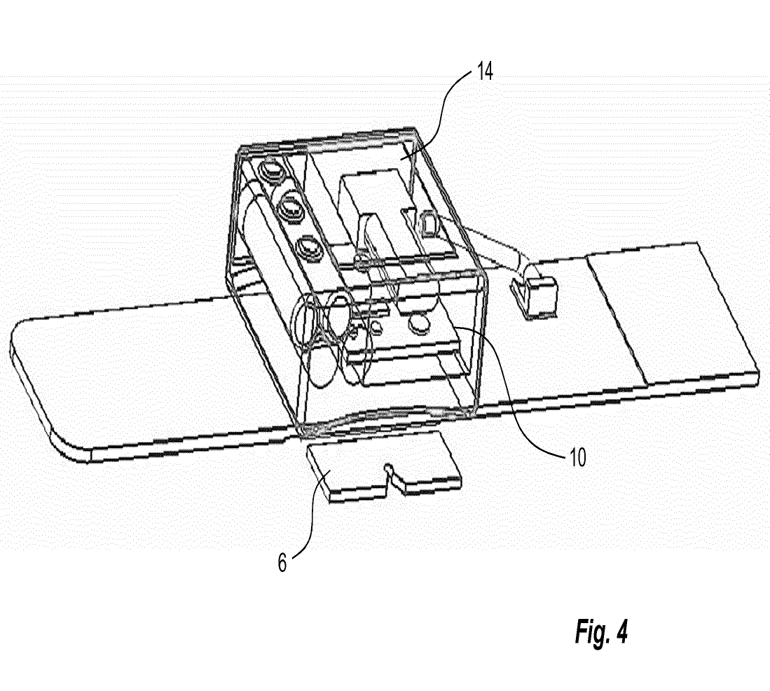

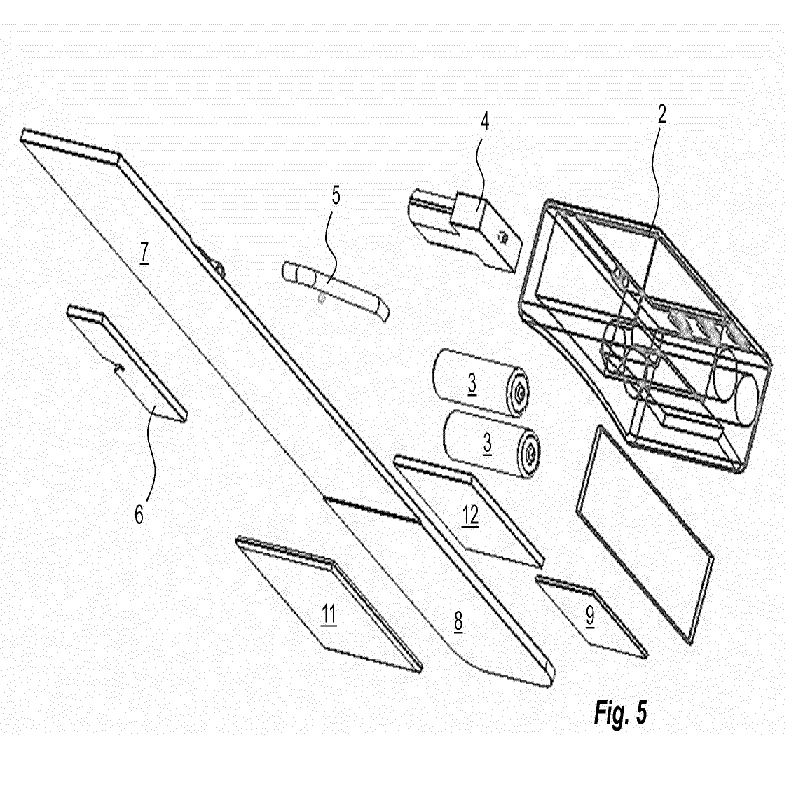

As depicted in FIGS. 4-5, the arrangement (1) additionally comprises a vein locator assist system (6) for locating the veins or arteries under the patient's skin (e.g. by IR spectroscopy). The locator vein system may be a modified type of vein locator system as disclosed in the publication WO2009/037432A1 and herein incorporated as reference. The vein locator assist system (6) may optionally be attachable to the arrangement. The vein locator assist system may comprise an infrared emitter/receiver that is capable of emit and receive infrared light and thus is capable of detecting reflected infrared radiation so as to identify a vein or an artery location beneath the patient's skin. This is because infrared radiation is reflected by tissue under the skin or is absorbed by blood in the veins or arteries. When the emitter/receiver detects a blood vessel, a suitable indication is produced, that could be audible or visual. The indicator portion or V-portion of the locator (6) allows marking of the patient's skin accordingly. Moreover, the casing is equipped with a display (14), or a mini screen, having visual or audible means. The display may be a touch display having features for starting, pausing, resetting, controlling inflation and/or deflation of the bladder, etc.

The strap tourniquet arrangement of the invention is not limited by the description but it may comprise additional features/elements having suitable functions among them several types of sensors e.g. temperature sensors; functionality buttons for starting, pausing, resetting, control inflate and/or deflate the bladder, etc.; a storage case over the casing for storing additional items (e.g. syringes, vials, pads, etc.); a communication module configured to transmit and/or receive information and/or signals directly and/or indirectly from the control unit to a smart device. This allows the caregiver/physician and/or the patient to utilize a WI-FI and/or Bluetooth and/or a mobile communication network capability to connect with any smart device. This is very useful specially if the patient is an ambidextrous and or has amputated arm, therefore remotely a caregiver/physician can trigger and control the different functionalities of the arrangement. Although preferred embodiments of the invention have been described, it is to be understood that these are by way of example only and that various modifications may be contemplated.

1.-10. (canceled)

11. A smart tourniquet arrangement for use in venipuncture operations, comprising:
a casing having a control unit;
a contact area arranged to the casing, the contact area being configured to contact a patient's skin and to be connected to the control unit;
a cuff arranged to the casing and having an inflatable bladder;
an adjustable strap arranged to the cuff for securing the cuff and the casing to the patient;
a pump contained in the casing, the pump being connected to the inflatable bladder and being controlled by the control unit;
a thermoelectric module contained in the casing, the thermoelectric module being controlled by the control unit and being connected to the contact area; and
at least one sensor contained in the casing for detecting blood pulse, the at least one sensor being controlled by the control unit;
wherein the control unit is configured to inflate and deflate the inflatable bladder in response to blood pulse for increasing or decreasing a pressure around an arm or around a leg and to heat the contact area for vasodilating a vein in the arm or leg, whereby the vein swells under the patient's skin for visual detection.

12. The arrangement of claim 11, further comprising a vein locator assist system.

13. The arrangement of claim 12, wherein the vein locator assist system is controlled by the control unit and comprises a portion that protrudes from the casing.

14. The arrangement of claim 11, wherein the control unit is configured to cool the contact area such that the cooled contact area causes a thermal analgesic effect reducing pain and constricts the vein.

15. The arrangement of claim 14, wherein the contact area is located on an outer surface of the casing.

16. The arrangement of claim 14, further comprising a vein locator assist system.

17. The arrangement of claim 16, wherein the vein locator assist system is controlled by the control unit and comprises a portion that protrudes from the casing.

18. The arrangement of claim 11, wherein the contact area is located on an outer surface of the casing.

19. The arrangement of claim 18, further comprising a vein locator assist system.

20. The arrangement of claim 19, wherein the vein locator assist system is controlled by the control unit and comprises a portion that protrudes from the casing.

21. The arrangement of claim 11, wherein the casing comprises a receiving element for receiving a pad that can be externally heated or cooled.

22. The arrangement of claim 21, wherein the pad is a hydrogel pad.

23. The arrangement of claim 11, further comprising a display or audio and/or visual indicators arranged on the casing and connected to the control unit.

24. The arrangement of claim 11, further comprising a communication module configured to communicate information between the control unit and a smart device.

25. The arrangement of claim 11, further comprising a storage case for the casing for storing at least one additional item.
